(19) United States
(12) Patent Application Publication
(10) Pub. No.: US 2008/0131329 A1
(43) Pub. Date: Jun. 5, 2008

(54) Portable Ultraviolet Germicidal Lamp with Safety Switch

(75) Inventors: Pei-Ling Lin, Taichung County (TW); Teng-Wen Nien, Taichung County (TW)

Correspondence Address: WPAT, PC, 7225 BEVERLY ST., ANNANDALE, VA, 22003, US

(73) Assignee: GOOTEN INNOLIFE CORPORATION, Taichung County (TW)

(21) Appl. No.: 11/565,407

(22) Filed: Nov. 30, 2006

(51) Int. Cl. G05D 99/00

(52) U.S. Cl. 422/105

(57) ABSTRACT

A portable ultraviolet (UV) germicidal lamp is disclosed, which is characterized by a safety switch configured inside a housing of the UV germicidal lamp that is electrically connected to a control circuit for controlling the UV germicidal lamp to be turned on and directing the same to emanate a UV ray downwardly while the UV germicidal lamp is positioned at a normal condition, and controlling the UV germicidal lamp to be turned off as the safety switch is being activated while the housing of the UV germicidal lamp is inclined by a specific angle and thus preventing the UV ray from shining upward.

### FIELD OF THE INVENTION

The present invention relates to a portable ultraviolet (UV) germicidal lamp, and more particularly, to a portable UV germicidal lamp having a safety switch that is capable of using the safety switch for preventing the UV germicidal lamp from shining a UV ray upwardly.

### BACKGROUND OF THE INVENTION

It is known that any conventional portable UV germicidal lamp is basically structured as a housing having a power source, a control circuit and a UV lamp formed therein while electrically connected to each other. By turning on a control switch of the control circuit, electricity of the power source is fed to the UV lamp for activating the same to discharge a UV sterilization light out of the housing through an opening formed thereon.

The goal of designing a portable UV germicidal lamp is to enable a user to carry the UV lamp easily and conveniently, so that the user can use the portable UV germicidal lamp to sterilize a toilet seat before using it for preventing contagious diseases.

However, the UV ray discharged out of the portable UV germicidal lamp is harmful as it is shined directly to a human body. Hence, an unwary user might accidentally tilt a conventional portable UV germicidal lamp that causing the head of the user to be shined by a harmful UV ray as there is usually no relating safety devices fitted in the conventional portable UV germicidal lamp. For preventing such accident, the design of those conventional portable UV germicidal lamps should be improved.

Moreover, as the control switch of a conventional portable UV germicidal lamp might be touched accidentally causing the UV lamp to be turned on when it is being carried around by a user in one's backpack, the user might take the UV lamp out of one's backpack unknowing that the UV lamp is on and thus cause the UV ray of the UV lamp to shine directly on the user. Therefore, the design of the control switch of those UV germicidal lamp should be improved as well.

### SUMMARY OF THE INVENTION

In view of the disadvantages of prior art, the primary object of the present invention is to provide a portable UV germicidal lamp having a safety switch that is capable of using the safety switch for preventing the UV germicidal lamp from shining a UV ray upward.

To achieve the above object, the present invention provides a portable UV germicidal lamp with a safety switch, being structured as a hollow housing having a power supply, a control circuit and a UV lamp formed therein while electrically connected to each other; by which as a control switch of the control circuit is turned on, electricity of the power supply is fed to the UV lamp for activating the same to discharge a UV sterilization light out of the housing through an opening formed thereon. Preferably, a handle is arranged on the housing at a position away from the opening; and the safety switch, being electrically connected to the control circuit, is fixedly disposed inside the housing in a manner that the safety switch will cause the control circuit to break and thus turn off the UV lamp as soon as the housing is tilted by a specific angle.

In a preferred embodiment, the safety switch is substantially a roll ball switch, that is structured by mounting two electrodes at a bottom of a hollow housing while disposing a steel ball between the two electrodes, by which the steel ball is positioned to have contact to the two electrodes while the UV germicidal lamp is positioned at a normal condition; and the steel ball is rolled away to cause a break between the two electrodes while the housing is tilted by the specific angle. Accordingly, as soon as the housing of the portable UV germicidal lamp is tilted by the specific angle, enabling the steel ball to roll away and causing an broken circuit to be formed between the two electrodes, the portable UV germicidal lamp is turned off and thus prevents the UV germicidal lamp from shining a UV ray upward.

Another object of the invention is to provide a portable UV germicidal lamp capable of preventing being turned on by mistake as it is being transport.

To achieve the above object, an additional indication lamp is configured in the control circuit of the aforesaid portable UV germicidal lamp in a manner that the first touch of the control switch of control circuit can only turn on the indication lamp and the UV lamp can only be turned on by the second touch of the control switch when the indication lamp is already on. Accordingly, the portable UV germicidal lamp can be prevented from being turned on by mistake as it is being transport.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 shows an appearance of a portable UV germicidal lamp according to the present invention.

FIG. 2 is a cross-sectional view of a portable UV germicidal lamp according to the present invention.

FIG. 3 shows a safety switch used in a portable UV germicidal lamp of the present invention.

FIG. 4 shows a portable UV germicidal lamp of the present invention as it is being tilted.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

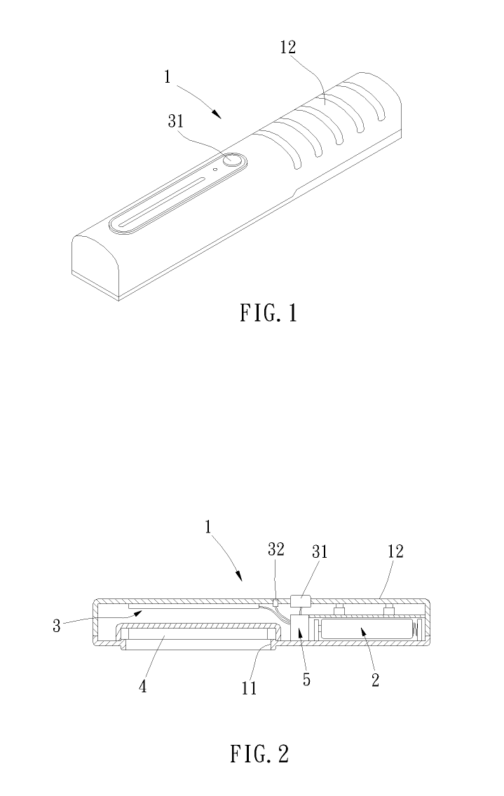

As seen in FIG. 1 to FIG. 4, a portable UV germicidal lamp is structured as a hollow housing 1 having a power supply 2, a control circuit 3 and a UV lamp 4 formed therein while electrically connected to each other; in which as a control switch 31 of the control circuit 3 is turned on, electricity of the power supply 2 is fed to the UV lamp 4 for activating the same to discharge a UV sterilization light downwardly out of the housing 1 through an opening 11 formed on the housing at a position corresponding to the UV lamp 4. In addition, a handle 12 is arranged on the housing 1 at a position away from the opening 11.

In a preferred embodiment of the invention, the power supply 2 can be a battery set, and the aforesaid control switch is arranged on the housing 1 at a position away from the opening 11, i.e. at the top of the housing proximate to the handle 12, so that the operation convenience of the portable UC germicidal lamp can be enhanced. Moreover, an indication lamp 32 is configured in the control circuit 3 of the aforesaid portable UV germicidal lamp and arranged on the housing 1 at a position proximate to the control switch 31 in a manner that the first touch of the control switch 31 can only turn on the indication lamp 32 and the UV lamp 4 can only be turned on by the second touch of the control switch 31 when the indication lamp 32 is already on.

In addition, a safety switch 5, being electrically connected to the control circuit 3, is fixedly disposed inside the housing 1 in a manner that the safety switch 5 will cause a broken circuit in the control circuit 3 and thus turn off the UV lamp 4 as soon as the housing 1 is tilted by a specific angle for preventing the UV germicidal lamp from shining a UV ray upward.

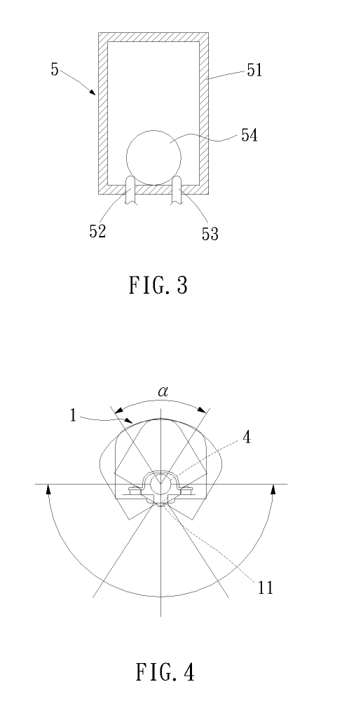

As seen in FIG. 3, the safety switch 5 is substantially a roll ball switch, that is structured by mounting two electrodes 52, 53 at a bottom of the hollow housing 51 while disposing a steel ball 54 between the two electrodes 52, 53, by which the steel ball 54 is positioned to have contact to the two electrodes 52, 53 while the UV germicidal lamp is positioned at a normal condition; and the steel ball 54 is rolled away to cause a broken circuit between the two electrodes 52, 53 while the housing 51 is tilted by the specific angle, and thus the portable UV germicidal lamp is turned off.

When a user is operating a portable UV germicidal lamp by holding the handle 12 thereof while maintaining the opening 11 of the housing 1 to face downward, the UV ray discharging out of the UV lamp 4 is maintained to shine generally directly downward although the angle thereof might be tilted from time to time. When the UV ray discharging out of the UV lamp 4 is maintained to shine generally directly downward, the safety switch 5 will maintain the conduction of the control circuit for enabling the portable UV germicidal lamp to operate normally.

However, when the tilting of the portable UV germicidal lamp exceeds a specific angle, as the angle a illustrated in FIG. 4 where the UV ray is likely to shine upward, the steel ball of the safety switch 5 will roll away from contacting to the two electrodes to cause a broken circuit, and thus the UV lamp 4 is turned off. Thereby, the portable UV germicidal lamp is turned off and thus prevents the UV germicidal lamp from shining a UV ray upward for causing damages to human body.

As the UV germicidal lamp is designed to be portable, a user might like to put such UV germicidal lamp in one's backpack or purse as the user is moving around. However, there can be more than just such UV germicidal lamp being stored in the backpack/purse that the control switch 31 of such UV germicidal lamp might be touched by those other objects by accident during the transportation. In order to prevent the portable UV germicidal lamp from being turned on by mistake as it is being transport, an indication lamp 32 is configured in the control circuit 3 of the aforesaid portable UV germicidal lamp in a manner that the first touch of the control switch 31 can only turn on the indication lamp 32 and the UV lamp 4 can only be turned on by the second touch of the control switch 31 when the indication lamp 32 is already on.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A portable ultraviolet (UV) germicidal lamp with a safety switch, comprising:
a hollow housing, for receiving a power supply, a control circuit and a UV lamp formed therein while electrically connected to each other; in which as a control switch of the control circuit is turned on, electricity of the power supply is fed to the UV lamp for activating the same to discharge a UV sterilization light out of the housing through an opening formed on the housing at a position corresponding to the UV lamp.
a handle, arranged on the housing at a position away from the opening; and
the safety switch, electrically connected to the control circuit while being fixedly disposed inside the housing in a manner that the safety switch will cause a broken circuit in the control circuit and thus turn off the UV lamp as soon as the housing is tilted by a specific angle.

2. The portable ultraviolet germicidal lamp of claim 1, wherein the safety switch is substantially a roll ball switch, being structured by mounting two electrodes at a bottom of a hollow housing while disposing a steel ball between the two electrodes, by which the steel ball is positioned to have contact to the two electrodes while the UV germicidal lamp is positioned at a normal condition; and the steel ball is rolled away to cause a broken circuit between the two electrodes while the housing is tilted by the specific angle.

3. The portable ultraviolet germicidal lamp of claim 1, wherein an additional indication lamp is configured in the control circuit of the portable UV germicidal lamp in a manner that the first touch of the control switch of control circuit can only turn on the indication lamp and the UV lamp can only be turned on by the second touch of the control switch when the indication lamp is already on.

4. The portable ultraviolet germicidal lamp of claim 1, wherein the indication lamp is disposed on the housing at a position away from the opening.
